(19) United States
(12) Patent Application Publication
(10) Pub. No.: US 2022/0047752 A1
(43) Pub. Date: Feb. 17, 2022

(54) BIOLOGICAL INDICATOR FOR DISINFECTION PROCESS CHALLENGE DEVICE

(71) Applicant: 3M INNOVATIVE PROPERTIES COMPANY, SAINT PAUL, MN (US)

(72) Inventors: ASSUMPTA A.G. BENNAARS-EIDEN, WOODBURY, MN (US); G. MARCO BOMMARITO, STILLWATER, MN (US); TONYA D. BONILLA, WOODBURY, MN (US); JODI L. CONNELL, SAINT PAUL, MN (US); RUTHANN R. DUDA, EAGAN, MN (US); FEDERICA SGOLASTRA, SAINT PAUL, MN (US); ANDREW W. VAIL, WOODBURY, MN (US)

(73) Assignee: 3M INNOVATIVE PROPERTIES COMPANY, SAINT PAUL, MN (US)

(21) Appl. No.: 16/316,407

(22) PCT Filed: Aug. 2, 2017
(86) PCT No.: PCT/IB2017/054737

§ 371 Date: Jan. 9, 2019

Related U.S. Application Data
(60) Provisional application No. 62/370,468, filed on Aug. 3, 2016.

(51) Int. Cl. A61L 2/28; C12Q 1/18; C12Q 1/22; C12Q 1/04; C12Q 1/6895

(52) U.S. Cl. CPC A61L 2/28; C12Q 1/18; C12Q 1/22; A61L 2202/24; C12Q 1/6895; A61L 2202/121; C12Q 1/04

(57) ABSTRACT

A method of verifying the efficacy of a disinfection process is provided. The method includes flowing a liquid disinfectant into an indicator device that has a plurality of indicator microorganisms disposed therein, the indicator microorganisms comprising or capable of producing a detectable enzyme activity; contacting the indicator microorganisms with the liquid disinfectant for a predetermined minimum first period of time at a first temperature; after contacting the indicator microorganisms with the liquid disinfectant for the first period of time at the first temperature, contacting the indicator microorganisms for a second period of time with a detection medium comprising a detection reagent; and during or after the second period of time, detecting a quantity of the detectable enzyme activity that was not inactivated by the contact with the disinfectant. The indicator device is also provided.

### BACKGROUND

Endoscopic procedures play a beneficial role in the prevention, diagnosis and treatment of disease. Endoscopy procedures are performed using complex, reusable, flexible instruments that, when inserted into the hollow body, may become heavily contaminated with patient biomaterial and microorganisms, including potential pathogens. Careful reprocessing of flexible endoscopes between patients is critical to reducing the risk of cross-contamination and the possible transmission of pathogens.

Flexible endoscopes are rated as semi-critical according to the Spaulding classification for medical devices and therefore it is required that these devices be decontaminated by high-level disinfection. Thus, it is recommended that both endoscopes and reusable accessories be frequently visually inspected in the course of their use and reprocessing, including before, during and after use, as well as after cleaning and before high-level disinfection. However, a visually based method of verification has severe limitations when applied to flexible endoscopes because the complex, narrow lumens in these devices cannot be directly visually inspected.

Automated endoscope reprocessors (AERs) are used to clean and disinfect flexible endoscopes to a level that mitigates transmission of pathogenic organisms and disease between patients who are subject to an endoscopic procedure. Typically, the only information available to a user is the parametric information provided by the AER equipment itself which consists primarily of time and temperature information. The AER does not monitor chemical parameters capable of establishing the effectiveness of the disinfection cycle. There is a need for improved biological indicators to monitor the efficacy of disinfection processes that use liquid disinfectants.

### SUMMARY

In general, the present disclosure relates to methods and devices for verifying the efficacy of a disinfection process. In particular, the present disclosure relates to methods and devices for verifying the efficacy of a disinfection process that contacts the items to be disinfected with a liquid disinfectant. The indicator devices of the present disclosure comprise a hollow body having an inlet, an outlet, and a plurality of indicator microorganisms disposed in the hollow body between the inlet and the outlet. The indicator devices confine the indicator microorganisms in a primary container using a first component that is configured to prevent unintentional egress of the indicator microorganisms from the primary container via the inlet of the device and a second component that is configured to prevent unintentional egress of the indicator microorganisms from the primary container via the outlet of the device. In any embodiment, the first component may comprise a microporous barrier or a normally-closed one-way valve. In any embodiment, the second component may comprise a microporous barrier. The first and second components prevent unintentional release of the indicator microorganisms out of the primary container. Indicator microorganisms that survive contact with the liquid disinfectant are detected by detecting an enzyme activity associated with the indicator microorganisms. Advantageously, self-contained indicator devices of the present disclosure include a detection medium therein.

In one aspect, the present disclosure provides a method of verifying the efficacy of a disinfection process. The method can comprise flowing a liquid disinfectant used in the disinfection process into an indicator device; wherein the indicator device comprises a plurality of indicator microorganisms that comprise or are capable of producing a detectable enzyme activity, and a primary container in which the indicator microorganisms are confined. The primary container can comprise an inlet, an outlet, a microorganism compartment in which the indicator microorganisms are disposed, and a liquid flow path extending from the inlet through the microorganism compartment to the outlet. The indicator device can be configured to permit ingress of the liquid disinfectant into the microorganism compartment. The indicator device can be configured to prevent egress of the indicator microorganisms from the microorganism compartment. Flowing the liquid disinfectant into the indicator device can comprise contacting the indicator microorganisms with the liquid disinfectant. The method further can comprise, contacting the indicator microorganisms with the liquid disinfectant for a predetermined minimum first period of time at a first temperature; after contacting the indicator device with the liquid disinfectant for the first period of time at the first temperature, contacting the indicator microorganisms for a second period of time with a detection medium comprising a detection reagent; and, during or after the second period of time, detecting a quantity of the detectable enzyme activity that was not inactivated by the contact with the disinfectant.

In any embodiment of the above method, the indicator device can indicate the disinfection process was efficacious when the quantity of the detectable enzyme activity that was not inactivated by the contact with the disinfectant is less than a predetermined amount of the detectable enzyme activity. In any embodiment of the above method, wherein the detectable enzyme activity, if present in the indicator device during the second period of time, converts the detection reagent from a first state to a second state; wherein detecting the detectable enzyme activity comprises detecting the second state of the detection reagent. In any embodiment of the above methods, contacting the indicator microorganisms with the liquid disinfectant can comprise contacting the indicator microorganism with the liquid disinfectant can comprise contacting a vegetative cell or a spore of the indicator microorganism with the liquid disinfectant. In any of the above embodiments, contacting the indicator microorganism with the liquid disinfectant can comprise contacting spore of a filamentous fungal microorganism with the liquid disinfectant. In any of the above embodiments of the method, contacting the spore of the filamentous fungal microorganism with a liquid disinfectant can comprise contacting a spore of a species selected from a group consisting of Aspergillus niger, Aspergillus brasiliensis, Aspergillus nidulans, Aspergillus oryzae, Fusarium solani, Fusarium verticillioides, Paecilomyces variotii, Scedosporium apiospermum, Trichophyton rubrum, and Penicillium camemberti, and a combination of spores of any two or more of the foregoing species.

In any of the above embodiments of the method, the indicator device can comprise a hollow body having a first end and a second end, wherein the plurality of indicator microorganisms is disposed in the indicator device between the first end and the second end, wherein flowing the liquid disinfectant into the indicator device can comprise flowing a portion of the liquid disinfectant through the hollow body from the first end to the second end. In any of the above embodiments, the method further can comprise contacting the indicator microorganisms with an effective amount of a neutralizing agent that inhibits biocidal activity of the liquid disinfectant.

In another aspect, the present disclosure provides an indicator device for verifying the efficacy of a liquid disinfection process. The indicator device can comprise a hollow body having at least one wall that forms a chamber, the hollow body comprising an inlet, an outlet, and a liquid flow path extending from the inlet to the outlet; a microorganism compartment disposed in the chamber; and a plurality of indicator microorganisms enclosed in the microorganism compartment. The microorganism compartment can have an interior volume that is defined in part by a first component that prevents unintentional passage of the indicator microorganisms out of the microorganism compartment via the inlet and a second component that prevents unintentional passage of the indicator microorganisms out of the microorganism compartment via the outlet. In any of the above embodiments, the second component can comprise a microporous barrier. In any of the above embodiments, the first component can comprise a first microporous barrier or a normally-closed one-way valve. In any of the above embodiments, the second component can comprise a second microporous barrier.

In any of the above embodiments, the indicator device further can comprise at least one closure attached thereto, wherein the at least one closure prevents fluid flow through the inlet or the outlet. In any one of the above embodiments, the first indicator device further can comprise a detection medium contained in a frangible closed vessel disposed in the hollow body. The detection medium may comprise a nutrient that facilitates germination or growth of the indicator microorganisms, a detection reagent, a neutralizing agent that inhibits biocidal activity of the liquid disinfectant, or a combination of any two or more of the foregoing components.

In any of the above embodiments of the indicator device, the plurality of indicator microorganisms can be vegetative cells or spores that comprise or are capable of producing a detectable enzyme activity. In any of the above embodiments wherein the indicator microorganism comprises a spore, the spore may be a spore of a species selected from a group consisting of Aspergillus niger, Aspergillus brasiliensis, Aspergillus nidulans, Aspergillus oryzae, Fusarium solani, Fusarium verticillioides, Paecilomyces variotii, Scedosporium apiospermum, Trichophyton rubrum, and Penicillium camemberti, and a combination of spores of any two or more of the foregoing species.

The terms “comprises” and variations thereof do not have a limiting meaning where these terms appear in the description and claims.

As used herein, “a,” “an,” “the,” “at least one,” and “one or more” are used interchangeably. Thus, for example, a detectable enzyme activity can be interpreted to mean “one or more” detectable enzyme activities.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is a block diagram of one embodiment of a method of verifying the efficacy of a disinfection process according to the present disclosure.

FIG. 2 is a cross-sectional schematic side view of one embodiment of an indicator device that is suitable for use in a method of verifying the efficacy of a disinfection process according to the present disclosure.

FIG. 3 is a cross-sectional schematic side view of the indicator device of FIG. 2 with closures affixed at the first and second ends.

FIG. 4 is a cross-sectional schematic side view of an alternative embodiment of an indicator device with a microorganism carrier that is suitable for use in a method of verifying the efficacy of a disinfection process according to the present disclosure.

FIG. 5 is a cross-sectional schematic side view of the indicator device of FIG. 4 with closures affixed at the first and second ends.

FIG. 6 is plan view of the microorganism carrier of the indicator device of FIG. 4.

FIG. 7 is a cross-sectional schematic side view of an alternative embodiment of an indicator device comprising a one-way valve, the device being suitable for use in a method of verifying the efficacy of a disinfection process according to the present disclosure.

FIG. 8 is a cross-sectional schematic side view of the indicator device of FIG. 7 with the one-way valve in an open configuration.

FIG. 9 is a cross-sectional schematic side view of the indicator device of FIG. 7 with closures affixed at the first and second ends.

FIG. 10 is a block diagram of one embodiment of a process for disinfecting an article.

FIG. 11 is a cross-sectional schematic view of one embodiment of a self-contained indicator device that is suitable for use in a method of verifying the efficacy of a disinfection process according to the present disclosure.

FIG. 12 is a cross-sectional schematic view of an alternative embodiment of a self-contained indicator device that is suitable for use in a method of verifying the efficacy of a disinfection process according to the present disclosure.

FIG. 13 is an exploded schematic side view of one embodiment of an indicator device that is suitable for use in a method of verifying the efficacy of a disinfection process according to the present disclosure.

FIG. 14 is a schematic side view of the indicator device of FIG. 13.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the device, to indicate or imply necessary or required orientations of the device, or to specify how the invention described herein will be used, mounted, displayed, or positioned in use.

The present disclosure generally relates to methods and devices to verifying the efficacy of a disinfection process.

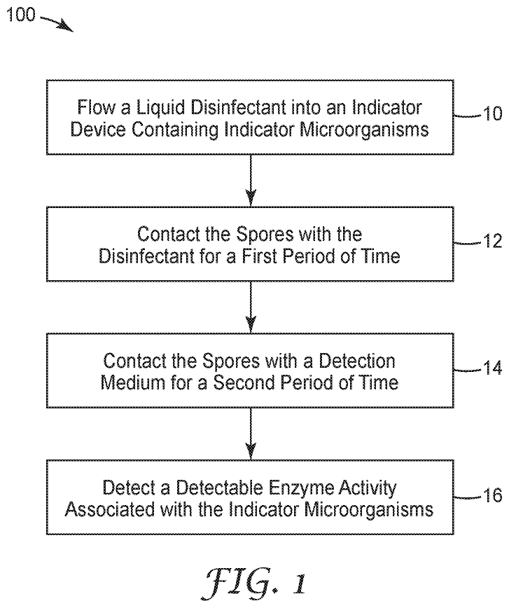

In one aspect, the present disclosure provides a method of verifying the efficacy of a disinfection process. FIG. 1 is a block diagram showing one embodiment of a method 100 of verifying the efficacy of a disinfection process according to the present disclosure.

The method 100 can comprise a step 10 of flowing a liquid disinfectant into an indicator device. In any embodiment, flowing the liquid disinfectant into the indicator device can comprise flowing the liquid disinfectant into the indicator device. In any embodiment, flowing the liquid disinfectant into the indicator device can comprise flowing at least a portion of the liquid disinfectant through the indicator device (e.g., flowing the liquid into an inlet, through the indicator device and out of an outlet, as described herein). Flowing the liquid disinfectant into the indicator device can comprise passively flowing the liquid disinfectant (e.g., via gravity) or capillary flow) or actively flowing the liquid disinfectant (e.g., via positive pressure applied to the liquid disinfectant and/or negative pressure applied to the outlet of indicator device).

During and/or after the step 10 of flowing the liquid disinfectant into the indicator device, the method 100 further comprises the step 12 of contacting indicator microorganisms with the liquid disinfectant for a predetermined minimum first period of time at a predetermined first temperature. Contacting the indicator microorganisms with the liquid disinfectant comprises contacting the indicator microorganisms with the liquid disinfectant in the indicator device.

A variety of indicator devices are suitable for use in a method according to the present disclosure, provided the indicator device comprises a) a plurality of indicator microorganisms (i.e., microorganisms that are susceptible to killing and/or inactivation by contact with the liquid disinfectant under suitable conditions), wherein the indicator microorganisms comprise or are capable of producing a detectable enzyme activity; b) a primary container that encloses the indicator microorganisms, the primary container having a chamber in which the indicator microorganisms are disposed, and c) at least two apertures (e.g., an inlet and an outlet) that form a flow path through the chamber. The inlet and the outlet, respectively, are determined by the direction of liquid flow through the device. A person having ordinary skill in the art will recognize that, in some embodiments of the present disclosure, liquid flow may be permitted in more than one direction (i.e., either of the at least two apertures can serve as the inlet or the outlet, respectively). In certain preferred embodiments, the primary container is configured to permit ingress of the liquid disinfectant into the chamber and to prevent egress of the indicator microorganisms out of the chamber.

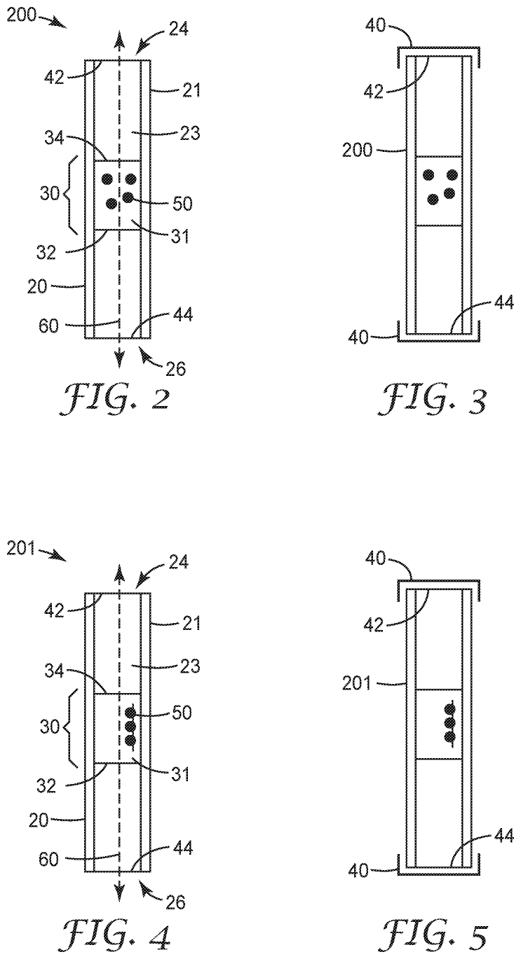

In addition to a method of verifying the efficacy of a disinfection process, the present disclosure provides indicator devices for use in the method. FIG. 2 shows a cross-sectional side view of one embodiment of an indicator device 200 suitable for use in a method of the present disclosure. The device 200 comprises a hollow body 20 that functions as a primary container in which the indicator microorganisms are confined. The hollow body 20 has at least one wall 21, a first end 24 and a second end 26 opposite the first end. The at least one wall 21 forms a chamber 23. The hollow body 20 is configured to permit ingress of a liquid disinfectant (not shown) into the chamber 23. Thus, in the illustrated embodiment of FIG. 2, the hollow body 20 comprises two spaced-apart apertures (e.g., an inlet 42 at the first end 24 of the hollow body and an outlet 44 at the second end 26 of the hollow body) that form a flow path 60 extending through the chamber 23. The inlet 42 can permit liquid disinfectant to move into the chamber 23. The outlet 44 can permit liquid disinfectant to move out of the chamber 23.

In the illustrated embodiment of FIG. 2, the device 200 includes a microorganism compartment 30 that has an interior volume 31 that is defined, in part by the at least one wall 21 of the hollow body 20 and by at least one microporous barrier (e.g., first microporous barrier 32). A “microporous barrier”, as used herein refers to a structure (e.g., a sheet-like structure) that is sufficiently porous to permit passage of liquid but the pores are sufficiently small or tortuous to prevent passage therethrough of the indicator microorganism (e.g., the filamentous fungal microorganisms disclosed herein). Disposed in the interior volume 31 of the microorganism compartment 30 is a plurality of indicator microorganisms 50. The first microporous barrier 32 prevents egress of the indicator microorganisms 50 from the chamber 23 via the outlet 44. In any embodiment (not shown), the first microporous barrier may be located at the outlet.

Optionally, the device 200 further comprises a second microporous barrier 34 disposed between the plurality of indicator microorganisms 50 and the inlet 42. In any embodiment (not shown), the second microporous barrier may be located at the inlet. In this embodiment, the microorganism compartment 30 is defined by the at least one wall, the first microporous barrier 32, and the second microporous barrier 34. The second microporous barrier 34, when present, prevents egress of the indicator microorganisms 50 from the chamber 23 via the inlet 42. Ingress of the liquid disinfectant into the chamber 23 of the device 200 facilitates passage of the liquid disinfectant into the interior volume 31 of the microorganism compartment 30, where the liquid disinfectant can contact the indicator microorganisms 50.

In any embodiment, the method of the present disclosure comprises contacting the plurality of indicator microorganisms with the liquid disinfectant in the indicator device. Non-limiting examples of liquid disinfectants that can be used in a method according to the present disclosure include glutaraldehyde, ortho-phthalaldehyde, and peroxyacetic acid. Contact between the indicator microorganisms and the liquid disinfectant can occur as the liquid disinfectant flows into and/or through the indicator device (e.g., as the liquid disinfectant flows into or though the hollow body 20 of the indicator device 200 of FIG. 2.

The hollow body 20 of the indicator device 200 can be fabricated using a material that can retain its structural integrity after contact with the liquid disinfectant. Preferably, the hollow body 20 can be sterilized (e.g., using heat, moist heat, ethylene oxide, or gamma irradiation) without losing its structural integrity and its ability to confine the indicator microorganisms 50 therein. Nonlimiting examples of suitable materials to fabricate the hollow body 20 include plastic, metal, glass, and combinations thereof. The hollow body 20 can be fabricated using processes (e.g., extrusion, injection molding) that are well known in the art. In any embodiment, the hollow body 20 may be constructed of a material (e.g., plastic or glass) that is optically transmissible in order to permit detection of microorganism growth in the hollow body.

The microorganism compartment 30 is configured to prevent egress of the indicator microorganisms 50 out of the interior volume 31. In one aspect, egress of the indicator microorganisms 50 out of the interior volume 31 is prevented, for example, by utilizing suitable materials, as described herein, for the microporous barrier(s). In another aspect, egress of the indicator microorganisms is prevented, for example, by attaching (e.g., via adhesive bonding or heat bonding) the microporous barriers (e.g., microporous barriers 32 and 34, respectively) to the at least one wall 21 of the hollow body 20 such that indicator microorganisms 50 cannot pass between the microporous barriers and the hollow body.

In any embodiment, the plurality of indicator microorganisms 50 may comprise spores of the indicator microorganism. In any embodiment, the indicator microorganisms may be vegetative cells of a prokaryotic microorganism or a eukaryotic microorganism. In any embodiment, the spores may be spores of a prokaryotic microorganism or a eukaryotic microorganism. In any embodiment, the spores may be spores of a filamentous fungal microorganism. In any embodiment, suitable spores of filamentous fungal microorganisms include, but are not limited to, spores of a genus selected from the group consisting of Aspergillus, Fusarium, Paecilomyces, Scedosporium, Trichophyton, and Penicillium, and a combination of spores of any two or more of the foregoing genera. In any embodiment, suitable spores include, but are not limited to, spores of a species selected from the group consisting of Aspergillus niger, Aspergillus brasiliensis, Aspergillus nidulans, Aspergillus oryzae, Fusarium solani, Fusarium verticillioides, Paecilomyces variotii, Scedosporium apiospermum, Trichophyton rubrum, and Penicillium camemberti, and a combination of spores of any two or more of the foregoing species.

The indicator microorganisms of the present disclosure comprise and/or are capable of producing (e.g., producing during or after germination) a detectable enzyme activity. In any embodiment, the detectable enzyme activity may be irreversibly inactivated by contact with an effective amount of the disinfectant. In any embodiment, inactivation of the detectable enzyme activity may correlate with loss of viability of the indicator microorganisms. Alternatively, or additionally, loss of the ability of the indicator microorganisms to produce the detectable enzyme activity may correlate with loss of viability of the indicator microorganisms. “Loss of viability of an indicator microorganism”, as used herein, refers to the inability of the indicator microorganism (which may be a spore) to reproduce to form two colony-forming units in the culture medium (or, in the case of a spore, to germinate into a vegetative cell. A liquid suspension of indicator microorganisms can be deposited onto the at least one wall of the hollow body, for example, before attaching the at least one microporous barrier to the at least one wall to form the microorganism compartment. Optionally, the liquid can be dried before attaching the at least one microporous barrier. Alternatively, a liquid suspension of indicator microorganisms can be deposited onto the at least one microporous barrier before the microporous barrier is attached to the at least one wall.

Optionally, the device 200 further can comprise closures 40 that are adapted to seal (e.g., by friction fit) the inlet 42 and/or the outlet 44 of the hollow body 20, as shown in FIG. 3. In use, the closures 40 can be repositioned, loosened, or removed, for example, in order to enable a liquid disinfectant to flow into or through the chamber 23 and contact the indicator microorganisms 50. Such closures 40 can prevent unwanted ingress of microorganisms into the chamber 23 during shipping, handling, or storage of the device 200. In addition, the closures 40 can be used in the detection step of the method of the present disclosure as described further hereinbelow. The closures 40 can be fabricated from rubber or plastic, for example, using processes (e.g., injection molding) that are known in the art.

The microporous barriers (e.g., microporous barrier 32 and/or 34) are configured to prevent passage of the indicator microorganisms 50 out of the chamber 23 of the hollow body 20. Thus, the microporous barriers 32 and 34 have pores that are configured to prevent the passage of the indicator microorganisms therethrough (e.g., the barriers have pores with a maximum cross-sectional size (e.g., diameter) that is smaller than the smallest dimension (e.g., diameter) of the indicator microorganisms or the pores are sufficiently tortuous to prevent passage of the indicator microorganisms completely through the microporous barrier). Microporous barrier 32 can be made from the same material or from a different material than microporous barrier 34. The nominal porosity of microporous barrier 32 and/or microporous barrier 34 can be, for example, less than or equal to 0.45 □m, less than or equal to 0.2 □m, or less than or equal to 0.1 □m.

In any of the above embodiments of an indicator device comprising a hollow body with an inlet and an outlet, the hollow body defines a liquid flow path extending from the inlet to the outlet, thereby providing fluid access to and egress from the chamber.

In any embodiment, the plurality of indicator microorganisms optionally can be disposed on a microorganism carrier. In these embodiments, the indicator microorganisms can be deposited (e.g., in a liquid coating that is optionally subsequently dried) on and/or into a microorganism carrier that is dimensioned to fit inside the microorganism compartment. FIG. 4 shows one embodiment of an indicator device 201 comprising a microorganism carrier 52. The indicator device 201 comprises a hollow body 20 that functions as a primary container in which the indicator microorganisms are confined. The hollow body 20 comprises at least one wall 21 that forms a chamber 23, as described herein. The hollow body 20 has a first end 24 and a second end 26. The first end 24 has spaced-apart apertures (e.g., inlet 42 and outlet 44) that provides fluid access into the chamber 23. Disposed in the hollow body 20 is a microorganism carrier 52 that has a plurality of indicator microorganisms 50 disposed thereon. Also disposed in the chamber 23, between the microorganism carrier 52 and the outlet 44, is a first microporous barrier 32. The first microporous barrier 32 is attached to the at least one wall 21 as described herein in order to prevent egress of the indicator microorganisms 50 out of the hollow body 20 via the outlet 44. Optionally, the indicator device may comprise a second microporous barrier 34 (e.g., disposed in the hollow body 20 between the indicator microorganisms 50 and the inlet 42. The construction of the hollow body 20 and the microporous barriers (microporous barriers 32 and 34, respectively) are as described for other embodiments of the other indicator devices disclosed herein.

The microorganism carrier 52 can be fabricated using a variety of suitable materials (e.g., a glass or metal sheet, a plastic film, a metal foil, a woven sheet-like, a nonwoven sheet-like material). The microorganism carrier 52 can be placed into chamber of the device, for example, before at least one of the microporous barriers (e.g., microporous barrier 32 and/or microporous barrier 34) is attached to the at least one wall 21 of the device 201.

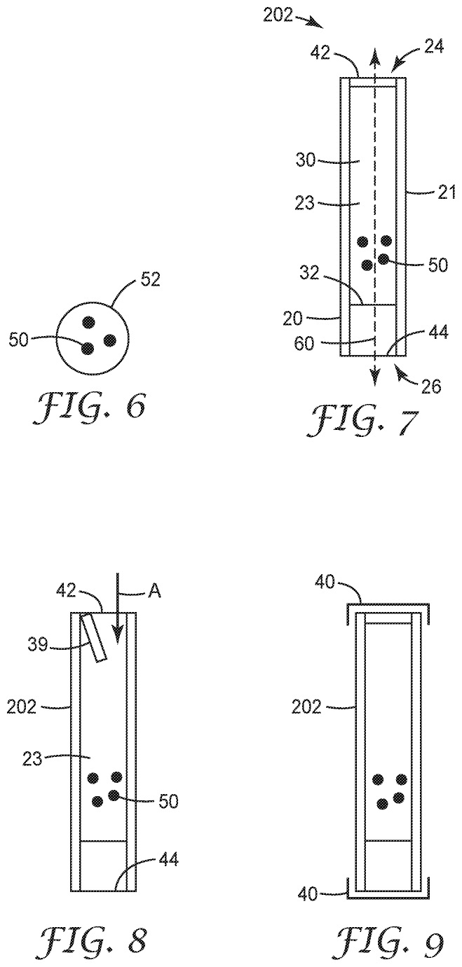

FIG. 6 shows a plan view of the microorganism carrier 52 of the indicator device 201 of FIG. 4. In the illustrated embodiment of FIG. 4, the microorganism carrier 52 has a circular shape that is dimensioned to fit in the chamber of the hollow body. It is contemplated that the microorganism carrier may be provided in any suitable shape (e.g., polygon, chevron, crescent, half-circle) that can be contained in the microorganism compartment 30.

The hollow body 20 of the indicator device 201 can be fabricated using any material that can retain its structural integrity during and after contact with the liquid disinfectant. Preferably, the hollow body 20 can be sterilized (e.g., using heat, moist heat, ethylene oxide, or gamma irradiation) without losing its structural integrity and its ability to confine the indicator microorganisms 50 therein. Nonlimiting examples of suitable materials include plastic, metal, glass, and combinations thereof. The hollow body 20 can be fabricated using processes (e.g., extrusion, injection molding) known in the art.

Optionally, the device 201 further comprises closures 40 that are adapted to seal (e.g., by friction fit) the inlet 42 and/or the outlet 44 of the hollow body 20, as shown in FIG. 5. In use, at least one of the closures 40 can be loosened or removed in order to enable a liquid disinfectant to flow into or through the chamber 23 and contact the indicator microorganisms 50. Such closures 40 can prevent unwanted ingress of microorganisms into the chamber 23 of the hollow body 20 during shipping, handling, or storage. In addition, the closures 40 can be used in the detection step of the method of the present disclosure as described further hereinbelow. The closures 40 can be fabricated from rubber or plastic, for example, using processes (e.g., injection molding) that are known in the art.

FIG. 7 shows an alternative embodiment of a suitable indicator device 202 according to the present disclosure. The device 202 comprises a hollow body 20 that functions as a primary container in which the indicator microorganisms are confined. The hollow body 20 has at least one wall 21, a first end 24 and a second end 26 opposite the first end. The at least one wall 21 forms a chamber 23. The hollow body 20 is configured to permit ingress of a liquid disinfectant (not shown) into the chamber 23. Thus, in the illustrated embodiment of FIG. 7, the hollow body 20 comprises two spaced-apart apertures (e.g., an inlet 42 at the first end 24 of the hollow body and an outlet 44 at the second end 26 of the hollow body) that form a flow path 60 extending through the chamber 23. The inlet 42 can permit liquid disinfectant to move into the chamber 23. The outlet 44 can permit liquid disinfectant to move out of the chamber 23.

The device 202 includes a microorganism compartment 30 that has an interior volume that is defined, in part by the at least one wall 21, the at least one microporous barrier (first microporous barrier 32), and a normally-closed one-way valve 39. Disposed in the microorganism compartment 30 is a plurality of indicator microorganisms 50. A first microporous barrier 32 prevents egress of the indicator microorganisms 50 from the chamber 23 via the outlet 44. In any embodiment (not shown), the first microporous barrier may be located at the outlet of the hollow body. A normally-closed one-way valve 39 permits one-way flow of a liquid (e.g., the liquid disinfectant) into the chamber 23 and prevents egress of the indicator microorganisms 50 from the chamber via the inlet 42. The normally-closed valve 39 is disposed between the indicator microorganisms 50 and the inlet 42.

In any embodiment, the method of the present disclosure comprises contacting the plurality of indicator microorganisms with the liquid disinfectant in the indicator device. Non-limiting examples of liquid disinfectants that can be used with the indicator device 202 in a method according to the present disclosure include glutaraldehyde, ortho-phthalaldehyde, and peroxyacetic acid. Contact between the indicator microorganisms and the liquid disinfectant can occur as the liquid disinfectant flows into and/or through the indicator device (e.g., as the liquid disinfectant flows into or though the hollow body 20 of the indicator device 202 of FIG. 7.

The hollow body 20 of the indicator device 202 can be fabricated using a material that can retain its structural integrity after contact with the liquid disinfectant. Preferably, the hollow body 20 can be sterilized (e.g., using heat, moist heat, ethylene oxide, or gamma irradiation) without losing its structural integrity and its ability to confine the indicator microorganisms 50 therein. Nonlimiting examples of suitable materials to fabricate the hollow body 20 include plastic, metal, glass, and combinations thereof The hollow body 20 can be fabricated using processes (e.g., extrusion, injection molding) that are well known in the art. In any embodiment, the hollow body 20 may be constructed of a material (e.g., plastic or glass) that is optically transmissible in order to permit detection of microorganism growth in the hollow body.

The microorganism compartment 30 is configured to prevent egress of the indicator microorganisms 50. In one aspect, egress of the indicator microorganisms 50 out of the microorganism compartment 30 is prevented by utilizing suitable materials, as described herein, for the first microporous barrier 32 and the normally-closed one-way valve 39. Egress of the indicator microorganisms 50 via the outlet 44 is prevented by attaching (e.g., via adhesive bonding or heat bonding) the first microporous barrier 32 to the at least one wall 21 of the hollow body 20 such that indicator microorganisms 50 cannot pass between the first microporous barrier and the hollow body. Egress of the indicator microorganisms 50 via the inlet 42 can be prevented by using a one-way valve 39 such as a duckbill-type valve, an umbrella valve, a cross-slit valve, a dome valve, a Bellville valve, for example, that is only open when liquid is flowing therethrough. The liquid flow is oriented so that any movement of the indicator microorganisms 50 through the body 20 would direct the indicator microorganisms toward the first microporous barrier, which would retain the indicator microorganisms in the hollow body.

FIG. 7 shows the indicator device 202 with the normally-closed valve 39 in a closed configuration. In this configuration, the valve 39 prevents egress of the indicator microorganisms 50 via the inlet 42 and the first microporous barrier 32 prevents egress of the indicator microorganisms 50 via the outlet 44.

FIG. 8 shows the indicator device 202 with the normally-closed one-way valve 39 in an open configuration. Movement of liquid (e.g., a liquid disinfectant) in the direction of arrow “A” can open the valve 39, thereby permitting movement of the liquid into (or through) the chamber 23 where the liquid can contact the indicator microorganisms 50.

In any embodiment, the plurality of indicator microorganisms 50 may comprise spores from a filamentous fungal microorganism, as described herein.

Optionally, the device 202 further comprises closures 40 that are adapted to seal (e.g., by friction fit) the inlet 42 and/or the outlet 44 of the hollow body 20, as shown in FIG. 9. In use, at least one of the closures 40 can be loosened or removed in order to enable a liquid disinfectant to flow into or through the chamber 23 and contact the indicator microorganisms 50. Such closures 40 can prevent unwanted ingress of microorganisms into the chamber 23 of the hollow body 20 during shipping, handling, or storage. In addition, the closures 40 can be used in the detection step of the method of the present disclosure as described further hereinbelow. The closures 40 can be fabricated from rubber or plastic, for example, using processes (e.g., injection molding) that are known in the art.

In any embodiment, a suitable indicator device of the present disclosure may comprise a film laminate (comprising two or more film components) having a liquid inlet, a liquid outlet, and a channel extending therebetween. The film components may be selected from the group consisting of a polymeric film, a metal foil, and combinations thereof. In any embodiment, one of the film components may be optically-transmissible to facilitate detection of a detection reagent, or product thereof, via optical interrogation methods that are known in the art. The indicator device may include a microorganism compartment housing indicator microorganisms in fluid communication with, and disposed between, the inlet and the outlet. The indicator device further may comprise a compartment holding detection medium, the compartment being in selective fluid communication with the microorganism compartment. An example of such an indicator device is shown and described in U.S. Patent Application No. 62/145,323 filed on Apr. 9, 2015, and entitled “PROCESS CHALLENGE DEVICE FOR AUTOMATED ENDOSCOPE REPROCESSOR”, which is incorporated herein by reference in its entirety. The formation of film laminates with channels and compartments disposed between the films is described, for example, in U.S. Pat. No. 7,026,168; which is incorporated herein by reference in its entirety.

Referring back to FIG. 1, a method 100 of the present disclosure comprises the step 12 of contacting the indicator microorganisms with the liquid disinfectant for a predetermined minimum first period of time at a predetermined first temperature. In any embodiment, the predetermined minimum first period of time can be a period of contact time between the indicator microorganisms and the disinfectant that is suitable to render at least 99% of the indicator microorganisms nonviable. In any embodiment, the predetermined minimum first period of time can be a period of disinfectant contact time that is suitable to render at least 99.9% of the indicator microorganisms nonviable. In any embodiment, the predetermined minimum first period of time can be a period of disinfectant contact time that is suitable to render at least 99.99% of the indicator microorganisms nonviable. In any embodiment, the predetermined minimum first period of time can be a period of disinfectant contact time that is suitable to render at least 99.999% of the indicator microorganisms nonviable. In any embodiment, the predetermined minimum first period of time can be a period of disinfectant contact time that is suitable to render at least 99.9999% of the indicator microorganisms nonviable. In any embodiment, the predetermined minimum first period of time can be a period of disinfectant contact time that is suitable to render at least 99.99999% of the indicator microorganisms nonviable.

In any embodiment, the predetermined minimum first period of time can be less than or equal to about 30 seconds, less than or equal to about 1 minute, less than or equal to about 2 minutes, less than or equal to about 3 minutes, less than or equal to about 4 minutes, less than or equal to about 5 minutes, less than or equal to about 6 minutes, less than or equal to about 7 minutes, less than or equal to about 8 minutes, less than or equal to about 9 minutes, less than or equal to about 10 minutes, less than or equal to about 11 minutes, less than or equal to about 12 minutes, less than or equal to about 13 minutes, less than or equal to about 14 minutes, less than or equal to about 15 minutes, less than or equal to about 17.5 minutes, less than or equal to about 20 minutes, less than or equal to about 22.5 minutes, less than or equal to about 25 minutes, less than or equal to about 30 minutes, less than or equal to about 35 minutes, less than or equal to about 40 minutes, less than or equal to about 45 minutes, less than or equal to about 50 minutes, less than or equal to about 55 minutes, less than or equal to about 60 minutes, less than or equal to about 70 minutes, less than or equal to about 80 minutes, less than or equal to about 90 minutes, less than or equal to about 100 minutes, less than or equal to about 110 minutes, or less than or equal to about 120 minutes.

In any embodiment, the predetermined first temperature can be about 20 degrees C. to about 50 degrees C. For example, for a disinfection process using ortho-phthalaldehyde (OPA) as the liquid disinfectant, the first temperature can be about 20 degrees C. to about 25 degrees C.; for a disinfection process using peracetic acid as the liquid disinfectant, the first temperature can be about 30 degrees C.; and for a disinfection process using glutaraldehyde as the liquid disinfectant, the first temperature can be about 35 degrees C. A person having ordinary skill in the art is aware that the efficacy of a particular disinfection process is related to the first temperature and the amount of contact time at the first temperature between the liquid disinfectant and the object to be disinfected.

After contacting the indicator microorganisms with the liquid disinfectant for the first period of time, the method 100 includes the step 14 of contacting the indicator microorganisms for a second period of time with a detection medium comprising a detection reagent. For example, the indicator device may be transferred aseptically to a vessel (e.g., a test tube, a flask, a bottle) containing any embodiment of the detection medium disclosed herein. The detection medium can flow into the indicator device, permitting detection of the enzyme activity according to the method of the present disclosure. Thus, the method 100 further comprises the step 16 of detecting a detectable enzyme activity associated with the indicator microorganisms. In an alternative embodiment, the detection medium may be introduced aseptically into the microorganism compartment (e.g., using a pipet or syringe) and the enzyme activity may be detected according to the method of the present disclosure. In another alternative embodiment, the detection medium may be provided in a self-contained indicator device as described herein and the medium can be brought into contact with the indicator microorganisms (as described below) after the indicator microorganisms are contacted with the liquid disinfectant.

The detection medium provides the proper aqueous environment (e.g., a detection reagent such as an enzyme substrate, for example at a suitable pH and at a concentration suitable for rapid detection (e.g., less than or equal to 8-hour detection, less than or equal to 4-hour detection, less than or equal to 3-hour detection, less than or equal to 2-hour detection, less than or equal to 1-hour detection, less than or equal to 30-minute detection, less than or equal to 20-minute detection, less than or equal to 10-minute detection) of any of the detectable enzyme activity that is not inactivated by contact with the liquid disinfectant). In any embodiment, the detection medium optionally may comprise a buffering agent and/or a pH indicator. The pH indicator, if present in the detection medium, can indirectly detect germination or growth of the indicator microorganism (e.g., spores).

In any embodiment, the detection medium further can comprise at least one nutrient that facilitates reproduction of the indicator microorganism (e.g., binary fission of a bacterium, germination and/or outgrowth of a spore). Additionally, or alternatively, the at least one nutrient comprises a nutrient that facilitates growth (i.e., the production of biomass including the detectable enzyme activity) of the indicator microorganism (e.g., a filamentous fungal microorganism that derives from germination of a spore). The at least one nutrient can include, for example, an amino acid, a polypeptide, a carbohydrate, a vitamin or cofactor, and a combination of any two of the foregoing nutrients.

In any embodiment, contacting the indicator microorganisms for a second period of time with a detection medium comprising a detection reagent comprises contacting the indicator microorganisms for a second period of time at a second predetermined temperature. The second predetermined temperature is selected to facilitate germination of a spore, growth of the vegetative cells, and/or to facilitate the detection of the detectable enzyme activity. A person having ordinary skill in the art is capable of selecting an appropriate second predetermined temperature based upon the identity of the species of the indicator microorganisms and/or the identity of the detectable enzyme activity. In general, indicator microorganisms used in indicator devices can be grown and the detectable enzyme activity can be detected at temperatures between 20-65 degrees C., inclusive. Filamentous fungal microorganisms suitable for use in a method of the present disclosure can be grown and the detectable enzyme activity can be detected at a temperature between 20-37 degrees C., inclusive. In any embodiment, the indicator microorganisms optionally may be heat-shocked prior to contacting the indicator microorganisms for a second period of time with a detection medium comprising a detection reagent.

The second period of time is a period of time that is suitable to facilitate growth and detection of the detectable enzyme activity. Suitable second periods of time include, for example, at least about 2 minutes and up to about 48 hours, at least about 2 minutes and up to about 18 hours, at least about 2 minutes and up to about 12 hours, at least about 2 minutes and up to about 8 hours, at least about 2 minutes and up to about 6 hours, at least about 2 minutes and up to about 5 hours, at least about 2 minutes and up to about 4 hours, at least about 2 minutes and up to about 3 hours, at least about 2 minutes and up to about 2 hours, at least about 2 minutes and up to about 90 minutes, at least about 2 minutes and up to about 1 hour, at least about 2 minutes and up to about 30 minutes, at least about 2 minutes and up to about 15 minutes, and at least about 2 minutes and up to about 10 minutes.

If the detectable enzyme activity is present in the indicator device during the second period of time (i.e., contact of the indicator microorganisms with the liquid disinfectant did not inactivate all of the detectable enzyme activity associated with the indicator microorganisms), the detectable enzyme activity converts the detection reagent from a first state to a second state that is detectably different from the first state. In any embodiment, the second state can be detected, for example, using a property selected from the group consisting of electromagnetic radiation absorption, electromagnetic radiation emission electrical conductivity, and electrical resistance. In any embodiment, the detection reagent may comprise a functional group (e.g., a chromophore or a fluorophore) that, after reacting with the detectable enzyme activity (or a reaction product thereof) to form the second state, can be detected (e.g., by a known absorbance or emission spectrum). Accordingly, the second state can be detected by its color, luminescence, or fluorescence, for example, and optionally can be measured using an appropriate automated detector. In any embodiment of the method, an appropriate positive and/or negative control indicator device can be processed in the automated detector to determine whether the disinfection process was efficacious. Alternatively or additionally, in any embodiment, a preset threshold value can be programmed into the automated detector to enable the detector to determine whether the disinfection process was efficacious.

In any embodiment, detecting the second state of the detection reagent can comprise detecting a first quantity of the second state at a first point in time and detecting a second quantity of the second state at a second point in time after the first point in time. A finding that the second quantity is substantially equivalent to the first quantity may be an indication that the disinfection process was efficacious. A finding that the second quantity is significantly greater (e.g., at least 10% greater, at least 20% greater, at least 50% greater, at least 75% greater, at least 100% greater, at least 200% greater, at least 300% greater, at least 400% greater, at least 500% greater, at least 600% greater, at least 800% greater, at least 1000% greater) than the first quantity may be an indication of the lack of efficacy of the disinfection process.

In any embodiment, the detection reagent used to detect the detectable enzyme activity is selected to correspond to the detectable enzyme activity. For example, if the detectable enzyme activity is □-glucosidase enzyme activity, the detection reagent is an enzyme substrate for □-glucosidase. If the detectable enzyme activity is an enzyme involved in the fermentation of a carbohydrate to an organic acid, for example, the detection reagent may be the carbohydrate and/or a pH indicator suitable for detecting accumulation of the organic acid in the detection medium.

A method according to the present disclosure comprises detecting the second state of the detection reagent that is produced by the detectable enzyme activity (or a reaction product thereof) of the plurality of indicator microorganisms in contact with the detection medium. In any embodiment, the second state can be detected, for example, using a property selected from the group consisting of electromagnetic radiation absorption, electromagnetic radiation emission electrical conductivity, and electrical resistance.

In an embodiment, detecting the second state of the detection reagent comprises detecting the second state of the detection reagent electrochemically (e.g., by measuring resistance, impedance, or conductivity of the detection medium). For example, in an embodiment of a method of the present disclosure, the indicator microorganisms may comprise and/or produce glucose oxidase enzyme. Thus, if the detection medium comprises glucose, the glucose oxidase enzyme activity can react with the glucose to form hydrogen peroxide, which can be detected electrochemically using a platinum or carbon electrode or it can be detected optically by reacting with luminol, for example, to produce chemiluminescence or by reacting with a chromogenic reagent (e.g., ferric iron and xylenol orange) to form a colored compound.

In some embodiments, the second state of the detection reagent can be detected manually (e.g., by visually observing a color change or fluorescence in the detection medium). In any of these embodiments, manual detection further can comprise comparing a color or fluorescence observable in the detection medium to a color standard or fluorescence standard in order to determine whether the disinfection process was efficacious.

If the liquid disinfectant used in the disinfection process has residual biocidal activity, contacting the indicator microorganisms with the liquid disinfectant will reduce the quantity of detectable enzyme activity present on and/or in the indicator microorganisms. The quantity of detectable enzyme activity will be reduced, for example, to less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 2%, less than or equal to 1%, less than or equal to 0.1%, less than or equal to 0.01%, less than or equal to 0.001%, or less than or equal to 0.0001% of the detectable enzyme activity of an identical indicator device that is not contacted with the liquid disinfectant.

In order to determine whether the disinfection process is efficacious, the quantity of detectable enzyme activity present in the indicator device after the indicator microorganisms are contacted with the liquid disinfectant can be compared to a predetermined amount of detectable enzyme activity. The predetermined amount can correlate with an expected amount of detectable enzyme activity present when at least 90%, at least 95%, at least 98%, at least 99%, at least 99.9%, at least 99.9%, at least 99.99%, at least 99.999%, or at least 99.9999% of the indicator microorganisms are inactivated (e.g., killed) by the liquid disinfectant. Thus, in any embodiment, the indicator device can indicate the disinfection process was efficacious when the quantity of the detectable enzyme activity that was not inactivated by the contact with the disinfectant is less than a predetermined amount of the detectable enzyme activity.

Any embodiment of the method of the present disclosure can be performed using one of a variety of disinfection chambers. For example, the disinfection chamber may be a sink (e.g., an open sink or a covered sink. In addition, the disinfection chamber may be a chamber specifically configured for high-level disinfection of medical devices. Thus, the chamber may be a chamber disposed in an automatic endoscope reprocessing (AER) machine. A person having ordinary skill in the art will recognize other chambers in which the method can be performed.

In any embodiment, the disinfection chamber can have a closed-loop liquid flow path (e.g., in an automated endoscope reprocessor). In these embodiment, the liquid disinfectant may be recirculated in the closed-loop flow path. Thus, in these embodiments, flowing the liquid disinfectant into the indicator device can comprise diverting (e.g., via a flexible connector tube) at least a portion of the liquid disinfectant from the liquid flow path to pass the portion into or through the indicator device and, optionally returning the portion (e.g., via a second connector tube) to the liquid flow path.

In any embodiment of a method according to the present disclosure, the indicator device can comprise a hollow body (e.g., the hollow body 20 of the indicator device 200 of FIG. 2) having a first end and a second end, wherein the plurality of indicator microorganisms is disposed in the hollow body between the first end and the second end. In these embodiments, flowing the liquid disinfectant into the indicator device can comprise flowing a portion of the liquid disinfectant through the hollow body from the first end toward the second end.

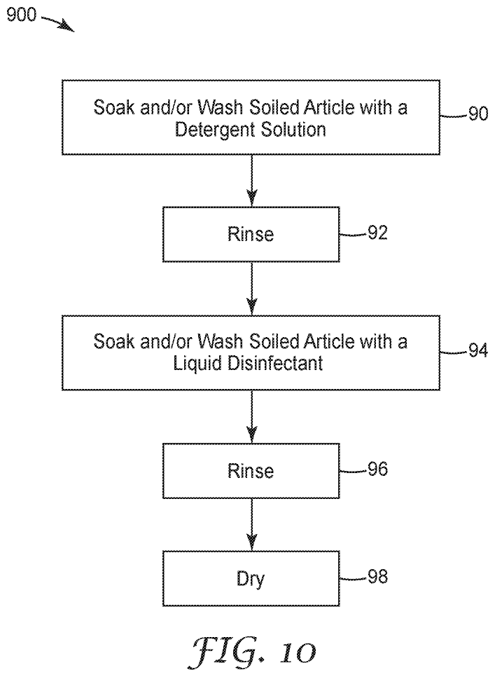

Processes for cleaning and disinfecting an article (e.g., an endoscope) with biological soil disposed thereon (and therein) typically involve a number of steps that reduce the amount of biological soil and, ultimately significantly reduce the number of viable microorganisms on and in the article. FIG. 10 shows one embodiment of a process 900 for removing biological soil from and disinfecting an article (e.g., a medical device). The process may include a step 90 of soaking and/or washing the soiled article with a detergent solution. The detergent facilitates solubilization and/or separation of biological soil from the surfaces (e.g., exterior and interior surfaces) of the article. Optionally, the detergent solution also may include one or more enzyme (e.g., a protease, a lipase, a glycosidase (e.g., amylase, cellulase)) to facilitate degradation and removal of biological soil from the surfaces of the article. After contacting the article with the detergent solution, the process 900 includes a step 92 of rinsing the article. Typically, the article is rinsed with water. After the rinse step 92, the process includes the step 94 of soaking and/or washing the article with a liquid disinfectant for a first period of time at a predetermined first temperature, as disclosed herein. Contact with the liquid disinfectant significantly reduces the number of viable microorganisms, if any, present on and/or in the article after the detergent wash step 90 and the rinse 92 step. After contacting the article with the liquid disinfectant for the first period of time, the process 900 may include a step 96 of rinsing the disinfected article. The second rinse step 96 functions to remove additional soil that may be present on surfaces of the article and also removes the liquid disinfectant from the article. Typically, the step 96 of rinsing the article is performed using water (e.g., filtered or otherwise purified water). After the second rinse step, the process 900 includes the step 98 of drying the article before it is re-used.

Thus, in any embodiment of verifying the efficacy of a disinfection process, optionally, the indicator device can be subjected to any one or all of the steps of the process in addition to the step that includes flowing the liquid disinfectant into the indicator device. Accordingly, in any embodiment, before flowing the liquid disinfectant into the indicator device, a method of verifying the efficacy of a disinfection process according to the present disclosure can include the step of flowing a liquid solution comprising a detergent into the indicator device, wherein said liquid solution does not include the liquid disinfectant. In any embodiment flowing the liquid solution comprising the detergent into the indicator device comprises flowing a liquid solution that comprises an enzyme (e.g., a protease, a lipase, a cellulase, an amylase, or combinations thereof) that facilitates degradation of biological soil into the indicator device. In any embodiment, flowing the liquid solution comprising the detergent into the indicator device comprises contacting the plurality of indicator microorganisms with the liquid solution comprising the detergent.

As described herein, processes (e.g., process 900 of FIG. 10) for cleaning and disinfecting an article include a rinse step after cleaning the article with a detergent solution. Thus, in any embodiment, after flowing the liquid solution comprising the detergent into the indicator device and before flowing the liquid disinfectant into the indicator device for the first period of time, a method of verifying the efficacy of a disinfection process according to the present disclosure optionally comprises the step of flowing a first rinse liquid into the indicator device (e.g., to remove the cleaning solution). In any embodiment, contacting the indicator device with the first rinse liquid comprises flowing the first rinse liquid into or through the indicator device. In any embodiment flowing the first rinse liquid into the indicator device comprises contacting the plurality of indicator microorganisms with the first rinse liquid. In any embodiment, the first rinse liquid can comprise water. In any embodiment, the first rinse liquid can consist essentially of water. In any embodiment, the first rinse liquid can consist of water.

After flowing the liquid disinfectant into or through the indicator device to contact the indicator microorganisms with the liquid disinfectant for the first period of time and before contacting the indicator microorganisms with the detection medium for a second period of time, in any embodiment, a method of the present disclosure optionally can comprise contacting the indicator device with a second rinse liquid (e.g., to remove the liquid disinfectant). In any embodiment, contacting the indicator device with the second rinse liquid comprises flowing the second rinse liquid into or through the indicator device. In any embodiment, contacting the indicator device with the second rinse liquid comprises contacting the plurality of indicator microorganisms with the second rinse liquid. In any embodiment, the second rinse liquid can comprise water. In any embodiment, the second rinse liquid can consist essentially of water. In any embodiment, the second rinse liquid can consist of water.

In one aspect, methods of the present disclosure are intended to assess the efficacy of the liquid disinfectant during contact with the indicator device for the first period of time at the predefined first temperature. However, if all of the liquid disinfectant is not removed from the indicator device shortly after the first period of time, any residual liquid disinfectant in contact with the indicator microorganisms after the first period of time may continue to inactivate the indicator microorganisms and/or the detectable enzyme activity. Thus, in some embodiments, it may be preferable to rinse and/or dilute (e.g., using purified water) any residual disinfectant remaining in the indicator device and/or to neutralize (i.e., inactivate) any residual liquid disinfectant remaining in the indicator device by contacting the indicator microorganisms with an effective quantity of a neutralizing agent after the disinfection step of the method. A “neutralizing agent”, as used herein, is a reagent that interferes with the biocidal activity of the liquid disinfectant. An “effective quantity” of neutralizing agent is a quantity that is sufficient to interfere with an amount of liquid disinfectant residue that would be reasonably expected in the indicator device after its contact with the liquid disinfectant and the rinse steps, if used, of the disinfection process. In any embodiment, the rinse step and/or the neutralization step preferably is initiated within about 60 minutes; preferably, less than about 10 minutes; more preferably, less than or equal to about 5 minutes; even more preferably, less than or equal to about 1 minute; and even more preferably, less than or equal to about 30 seconds after the indicator microorganisms are contacted with the liquid disinfectant for the first period of time.

In any embodiment, a method according to the present disclosure further comprises providing an indicator device having the features of any embodiment of the suitable indicator devices disclosed herein.

In any embodiment, contacting the device with the liquid disinfectant can comprise simultaneously contacting a medical device with the liquid disinfectant for the first period of time at the first temperature. Thus, the indicator device can be processed at the same time, and with the same liquid disinfectant, as the medical device. In these embodiment, the indicator device can be used to assess the efficacy of the disinfection of the medical device.

In any embodiment, the indicator device used in a method according to the present disclosure can be a self-contained biological indicator device. The self-contained biological indicator device includes a nutrient medium and an indicator reagent therein, so that any indicator microorganisms that survive contact with the liquid disinfectant can be cultured and detected in the self-contained indicator device.

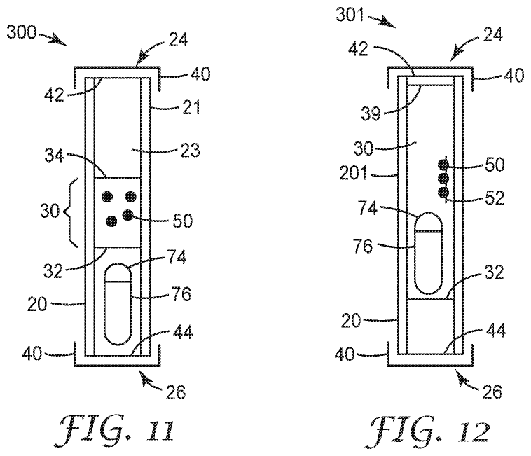

FIG. 11 shows one embodiment of a self-contained biological indicator device 300 that is suitable for use in a method of verifying the efficacy of a disinfection process according to the present disclosure. The indicator device 300 comprises a hollow body 20 with a first end 24, a second end 26, an inlet 42, and an outlet 44, each as described hereinabove. The hollow body 20 includes a microorganism compartment 30 that is defined in part by microporous barriers 32 and 34. The microorganism compartment 30 encloses a plurality of indicator microorganisms 50 in the hollow body 20. In addition, the indicator device 300 includes closures 40 that are configured to seal (e.g., by friction fit) the first end 24 and the second end 26 of the hollow body 20 after the liquid disinfectant (not shown) has contacted the indicator microorganisms 50. Thus, in any embodiment, the closures 40 may be repositioned, loosened, or removed, for example, from the first and second end of the hollow body 20 during the period of contact between the indicator device 300 and the liquid disinfectant (or other liquids such as rinse solutions, for example, that are flowed into or through the indicator device). Subsequently, the closures 40 may be tightened or re-affixed to the hollow body 20; optionally, after the liquid disinfectant is substantially removed (e.g., drained) from the hollow body.

Also included in the self-contained biological indicator device 300 is a frangible closed vessel 74 (e.g., a glass ampoule) that contains a detection medium 76. The detection medium 76 comprises the detection reagent selected to detect the detectable enzyme activity of the method of the present disclosure and, optionally, a nutrient to facilitate growth and/or germination of the indicator microorganisms. The detectable enzyme activity can be any detectable enzyme activity associated with the indicator microorganism (e.g., including spores or/or the vegetative cells derived therefrom. Non-limiting examples of suitable detectable enzyme activities include an enzyme activity selected from the group consisting of xylanase, cellulase, □-galactosidase, □-galactosidase, □-glucuronidase, phosphatase, □-glucosidase, □-glucosidase, amylase, glucose oxidase, laccase, and a combination of any two or more of the foregoing enzyme activities. Optionally, the detection medium further comprises an effective amount of a neutralizing agent as described herein.

FIG. 12 shows an alternative embodiment of a self-contained biological indicator device 301 that is suitable for use in a method of verifying the efficacy of a disinfection process according to the present disclosure. The indicator device 301 comprises a hollow body 20 with a first end 24, a second end 26, an inlet 42, and an outlet 44, each as described hereinabove. The hollow body 20 includes a microorganism compartment 30 that is defined in part by a first microporous barrier 32 and a normally-closed one-way valve 39. The microorganism compartment 30 encloses a plurality of indicator microorganisms 50 disposed on a microorganism carrier 52 as described herein. In addition, the indicator device 301 includes closures 40 that are configured to seal (e.g., by friction fit) the second end 26 and, optionally, the first end 24 of the hollow body 20 after the liquid disinfectant (not shown) has contacted the indicator microorganisms 50. Thus, in any embodiment, the closures 40 may be repositioned, loosened, or removed, for example, from the first and second end of the hollow body 20 during the period of contact between the indicator device 300 and the liquid disinfectant (or other liquids such as rinse solutions, for example, that are flowed into or through the indicator device). Subsequently, the closures 40 may be tightened or re-affixed to the hollow body 20; optionally, after the liquid disinfectant is substantially removed (e.g., drained) from the hollow body.

Also included in the self-contained biological indicator device 301 is a frangible closed vessel 74 (e.g., a glass ampoule) that contains a detection medium 76. The detection medium 76 comprises the detection reagent selected to detect the detectable enzyme activity of the method of the present disclosure and, optionally, a nutrient to facilitate growth and/or germination of the indicator microorganisms. The detectable enzyme activity can be any detectable enzyme activity associated with the indicator microorganism (including, for example, spores or the vegetative cells derived therefrom). Non-limiting examples of suitable detectable enzyme activities include an enzyme activity selected from the group consisting of xylanase, cellulase, □-galactosidase, □-galactosidase, □-glucuronidase, phosphatase, □-glucosidase, □-glucosidase, amylase, glucose oxidase, laccase, and a combination of any two or more of the foregoing enzyme activities. Optionally, the detection medium further comprises an effective amount of a neutralizing agent as described herein.

In use, the self-contained indicator devices are configured to permit flow of the liquid disinfectant into or through the hollow body and into contact with the indicator microorganisms. In any embodiment, “configured to permit flow of the liquid disinfectant into the hollow body” can comprise loosening or removing a closure, if present, to provide liquid access into the hollow body of the device. After contacting the indicator microorganisms with the liquid disinfectant for the first period of time, optionally, the liquid disinfectant can be allowed to drain from the device. One or more closure is then reattached and/or tightened to seal the hollow body of the device against liquid leakage and the contents of the sealed vessel (e.g., glass ampoule) are released (e.g., by applying force to the at least one wall to crush the ampoule) to bring the indicator microorganisms into liquid contact with the detection medium. The indicator microorganisms are then contacted with the detection reagent of the detection medium for a second period of time to permit the detection reagent to be converted from a first state to a second state by an enzyme activity of the indicator microorganisms, as described hereinabove. Subsequently, the second state is detected (e.g., after placing the housing into a detector) as described herein.

### EXEMPLARY EMBODIMENTS

Embodiment A is a method of verifying the efficacy of a disinfection process, comprising:
flowing a liquid disinfectant into an indicator device;
   wherein the indicator device comprises:
      a primary container comprising an inlet, an outlet, a microorganism compartment, and a liquid flow path extending from the inlet through the microorganism compartment to the outlet;
      a plurality of indicator microorganisms that comprise or are capable of producing a detectable enzyme activity, the indicator microorganisms being disposed in the microorganism compartment; and
      wherein the indicator device is configured to permit ingress of the liquid disinfectant into the microorganism compartment;
      wherein the indicator device is configured to prevent egress of the indicator microorganisms from the microorganism compartment;
   wherein flowing the liquid disinfectant into the indicator device comprises contacting the indicator microorganisms with the liquid disinfectant;
contacting the indicator microorganisms with the liquid disinfectant for a predetermined minimum first period of time at a first temperature;
after contacting the indicator microorganisms with the liquid disinfectant for the first period of time at the first temperature, contacting the indicator microorganisms for a second period of time with a detection medium comprising a detection reagent; and
during or after the second period of time, detecting a quantity of the detectable enzyme activity that was not inactivated by the contact with the disinfectant.

Embodiment B is the method of Embodiment A, wherein the indicator device indicates the disinfection process was efficacious when the quantity of the detectable enzyme activity that was not inactivated by the contact with the disinfectant is less than a predetermined amount of the detectable enzyme activity.

Embodiment C is the method of Embodiment A or Embodiment B, wherein the detectable enzyme activity, if present in the indicator device during the second period of time, converts the detection reagent from a first state to a second state; wherein detecting the detectable enzyme activity comprises detecting the second state of the detection reagent.

Embodiment D is the method of any one of the preceding Embodiments, wherein the plurality of indicator microorganisms comprises a plurality of vegetative cells.

Embodiment E is the method of any one of Embodiments A through C, wherein the plurality of indicator microorganisms comprises a plurality of spores.

Embodiment F is the method of Embodiment E, wherein contacting the indicator microorganism with the liquid disinfectant comprises contacting an indicator microorganism of a genus selected from the group consisting of Aspergillus, Fusarium, Paecilomyces, Scedosporium, Trichophyton, and Penicillium.

Embodiment G is the method of Embodiment F, wherein contacting the indicator microorganism with a liquid disinfectant comprises contacting an indicator microorganism of a species selected from the group consisting of Aspergillus niger, Aspergillus brasiliensis, Aspergillus nidulans, Aspergillus oryzae, Fusarium solani, Fusarium verticillioides, Paecilomyces variotii, Scedosporium apiospermum, Trichophyton rubrum, and Penicillium camemberti.

Embodiment H is the method of any one of the preceding Embodiments, wherein contacting the indicator microorganisms for a second period of time with a detection medium comprises contacting the indicator microorganisms with at least one nutrient that facilitates growth of the indicator microorganisms and/or, when the indicator microorganism is a spore, the effective amount of the at least one nutrient facilitates germination or outgrowth of the spore and/or growth of a vegetative cell derived from the spore.

Embodiment I is the method of any one of the preceding Embodiments, wherein contacting the indicator device with a liquid disinfectant comprises contacting the indicator device with a liquid disinfectant selected from the group consisting of glutaraldehyde, ortho-phthalaldehyde, and peroxyacetic acid.

Embodiment J is the method of any one of the preceding Embodiments, wherein detecting the second state of the detection reagent that is produced by the detectable enzyme activity comprises detecting a product of an enzyme activity selected from the group consisting of xylanase, cellulase, □-galactosidase, □-galactosidase, □-glucuronidase, phosphatase, □-glucosidase, □-glucosidase, amylase, glucose oxidase, laccase, and a combination of any two or more of the foregoing enzyme activities.

Embodiment K is the method of any one of the preceding Embodiments, wherein detecting the second state of the detection reagent that is produced by the detectable enzyme activity comprises detecting the second state using a property selected from the group consisting of electromagnetic radiation absorption, electromagnetic radiation emission, and electrochemical properties.

Embodiment L is the method of any one of the preceding Embodiments, wherein contacting the indicator device with the liquid disinfectant comprises contacting the device in a disinfection chamber.

Embodiment M is the method of Embodiment L, wherein the disinfection chamber has a closed-loop liquid flow path, wherein the liquid disinfectant flowed into the indicator device is recirculated in the closed-loop flow path.

Embodiment N is the method of Embodiment L, wherein the disinfection chamber has a closed-loop liquid flow path, wherein the liquid disinfectant is recirculated in the closed-loop flow path, wherein contacting the indicator device with the liquid disinfectant comprises diverting a portion of the liquid disinfectant from the closed-loop flow path into the indicator device.

Embodiment O is the method of Embodiment N, further comprising returning at least a fraction of the portion back to the closed-loop flow path after the fraction flows into the indicator device.

Embodiment P is the method of any one of the preceding claims, further comprising:
before flowing the liquid disinfectant into the indicator device, flowing a liquid solution other than the liquid disinfectant into the indicator device, wherein the liquid solution comprises a detergent.

Embodiment Q is the method of Embodiment P, wherein flowing the liquid solution into the indicator device comprises flowing a liquid solution that comprises an enzyme into the indicator device.

Embodiment R is the method of Embodiment Q, wherein flowing the liquid solution that comprises an enzyme into the device includes flowing a liquid solution that comprises an enzyme selected from the group consisting of a protease, a lipase, a glycosidase, an amylase, a cellulase, and a combination of any two or more of the foregoing enzymes into the indicator device.

Embodiment S is the method of any one of Embodiments P through R, wherein flowing the liquid solution into the indicator device comprises contacting the plurality of indicator microorganisms with the liquid solution.

Embodiment T is the method of any one of Embodiments P through S, further comprising:
after flowing the liquid solution into the indicator device and before flowing the liquid disinfectant into the indicator device, flowing a first rinse liquid into the indicator device.

Embodiment U is the method of Embodiment T, wherein flowing the first rinse liquid into the indicator device comprises contacting the plurality of indicator microorganisms with the first rinse liquid.

Embodiment V is the method of any one of the preceding Embodiments, further comprising:
after flowing the liquid disinfectant into the indicator device and before contacting the indicator microorganisms with the detection medium for a second period of time, flowing a second rinse liquid into the indicator device to remove at least a portion of the liquid disinfectant.

Embodiment W is the method of Embodiment V, wherein flowing the second rinse liquid into the indicator device comprises contacting the plurality of indicator microorganisms with the second rinse liquid.

Embodiment X is the method of any one of the preceding Embodiments, further comprising:
after flowing the liquid disinfectant into the indicator device, contacting the indicator microorganisms with an effective amount of a neutralizing agent that inhibits biocidal activity of the liquid disinfectant.

Embodiment Y is the method of Embodiment X, wherein contacting the indicator microorganisms with the detection medium comprises contacting the indicator microorganisms with the effective amount of the neutralizing agent.

Embodiment Z is the method of any one of the preceding Embodiments, wherein detecting the second state of the detection reagent comprises detecting a quantity of the second state.

Embodiment AA is the method of any one of the preceding Embodiments, wherein detecting the second state of the detection reagent comprises detecting a first quantity of the second state at a first point in time and detecting a second quantity of the second state at a second point in time after the first point in time.

Embodiment AB is the method of any one of the preceding Embodiments, further comprising providing the indicator device.

Embodiment AC is the method of any one of the preceding Embodiments, wherein flowing the liquid disinfectant into the indicator device comprises simultaneously contacting a medical device with the liquid disinfectant for the first period of time at the first temperature.

Embodiment AD is an indicator device for verifying the efficacy of a liquid disinfection process, the indicator device comprising:
a hollow body having at least one wall that forms a chamber, an inlet, an outlet, and a liquid flow path that extends from the inlet to the outlet;
a microorganism compartment disposed in the chamber; and
a plurality of indicator microorganisms enclosed in the microorganism compartment;
wherein the microorganism compartment has an interior volume that is defined in part by a first component that prevents unintentional passage of the indicator microorganisms out of the microorganism compartment via the inlet and a second component that prevents unintentional passage of the indicator microorganisms out of the microorganism compartment via the outlet.

Embodiment AE is the indicator device of Embodiment AD, wherein the first component comprises a first microporous barrier.

Embodiment AF is the indicator device of Embodiment AD or Embodiment AE, wherein the second component comprises a second microporous barrier.

Embodiment AG is the indicator device of Embodiment AD or Embodiment AF, wherein the first component comprises a normally-closed one-way valve.

Embodiment AH is the indicator device of any one Embodiments AD through AG, further comprising at least one closure attached thereto, wherein the at least one closure is configured to prevent fluid flow through the inlet or the outlet.

Embodiment AI is the indicator device of any one of Embodiments AD through AH, further comprising a detection medium contained in a frangible closed vessel disposed in the hollow body.

Embodiment AJ is the indicator device of any one of Embodiments AD through AI, wherein the plurality of indicator microorganisms comprises a plurality of vegetative cells.

Embodiment AK is the indicator device of any one of Embodiments AD through AI, wherein the plurality of indicator microorganisms comprises a plurality of spores.

Embodiment AL is the indicator device of Embodiment AK, wherein the indicator microorganism is of a genus selected from the group consisting of Aspergillus, Fusarium, Paecilomyces, Scedosporium, Trichophyton, and Penicillium.

Embodiment AM is the indicator device of Embodiment AL, wherein the indicator microorganism is of a species selected from the group consisting of Aspergillus niger, Aspergillus brasiliensis, Aspergillus nidulans, Aspergillus oryzae, Fusarium solani, Fusarium verticillioides, Paecilomyces variotii, Scedosporium apiospermum, Trichophyton rubrum, and Penicillium camemberti.

Embodiment AN is the indicator device of any one of the Embodiments AI through AM, wherein the detection medium comprises an effective amount of at least one nutrient that facilitates germination or growth of the indicator microorganisms and/or, when the indicator microorganism is a spore, the effective amount of the at least one nutrient facilitates growth of a vegetative cell derived from the spore.

Embodiment AO is the indicator device of Embodiment AI, wherein the detection medium comprises a detection reagent.

Embodiment AP is the indicator device of Embodiment AO, wherein the detection reagent comprises an enzyme substrate for detecting an enzyme activity selected from a group consisting of xylanase, cellulase, □-galactosidase, □-galactosidase, □-glucuronidase, phosphatase, □-glucosidase, □-glucosidase, amylase, glucose oxidase, laccase, and a combination of any two or more of the foregoing enzyme activities.

Embodiment AQ is the indicator device of any one of Embodiments AD through AP, wherein the first microporous barrier and/or the second microporous barrier comprises a membrane filter.

Embodiment AR is the indicator device of any one of Embodiments AD through AQ, wherein the plurality of indicator microorganisms is disposed on or in a microorganism carrier that is disposed in the microorganism compartment.

Embodiment AS is the indicator device of any one of Embodiment AD through AR, wherein the hollow body consists essentially of a laminate of at least two films.

Embodiment AT is the indicator device of Embodiment AS, wherein one film of the at least two films comprises a thermoplastic polymer.

Embodiment AU is the indicator device of Embodiment AS, wherein one film of the at least two films consists essentially of a thermoplastic polymer.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

### Example 1

### Flow-Through Indicator Device for Liquid Disinfection Process

Preparation of Spores of an Indicator Microorganism.

Aspergillus brasiliensis (ATCC 16404) was inoculated onto the surface of a potato dextrose agar culture and incubated 14 days at 25-30° C. After the incubation period, the surface of the plate was flooded with an aqueous solution of glycerol (10% v/v) and Tween 20 detergent (0.05% v/v). The viable fungal spore population was determined by standard plate counting method using PETRIFILM™ Rapid Yeast and Mold Count Plates (RYM) obtained from 3M Company (St. Paul, Minn.). The plates were incubated at 28-30° C. for 2-3 days. The resulting spore suspension was stored at −80 degrees C. until use.

The spore suspension was diluted in water to a concentration of approximately 10⁸ spores/mL. Ten microliters (approximately 1×10⁶ spores) of the diluted spore suspension was pipetted onto a circular disc (approximately 0.25 inch (6.4 mm) diameter) nonwoven carrier material (Sontara® grade 8005 nonwoven material obtained from Dupont (Candler, N.C.)). The spore-coated discs were allowed to dry for 24 hours at room temperature before assembling the indicator device.

Assembly of the Indicator Device.

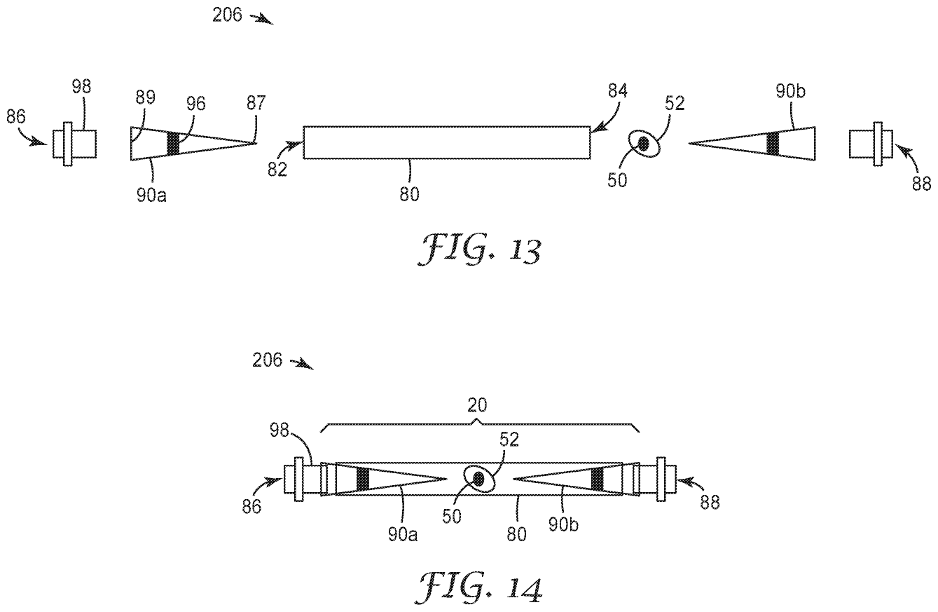

Indicator devices were made by inserting aerosol-filter pipet tips (Part #16466-008; sterile aerosol 1000 uL pipet tips; VWR; Radnor, Pa.) into a piece of hollow tubing (Part #14-169-7C; PVC tubing, 0.25-inch (6.4 mm) inner diameter; Fisher Scientific; Waltham, Mass.), as shown in FIGS. 13-14. The hollow tubing and pipet tips formed the hollow body 20 of the indicator device 206. The pipet tips (first pipet tip 90a and second pipet tip 90b, respectively) comprise a small opening 87, a large opening 89, and an aerosol filter (microporous barrier 32) lodged in the pipet tip between the small-opening and the large-opening. The small-opening 87 of the first pipet tip 90a was inserted into the first end 82 of the tubing 80 until the first pipet tip was tightly engaged in the tubing. A microorganism carrier (disc) 52 with a plurality of indicator microorganisms 50 (spores) coated thereon was subsequently inserted into the second end 84 of the tubing 80. Then, the small-opening of the second pipet tip 90b was inserted into the second end 84 of the tubing 80 until the second pipet tip was tightly engaged in the tubing. Optional Luer connectors 98 (Part #45500-20, Female luer thread style to 500 series, 0.25-inch (6.4 mm) inner diameter, natural polypropylene; Cole Parmer; Vernon Hills, Ill.) were inserted into the large opening 89 of each pipet tip to complete the assembly of the device. The Luer connectors 98 can be used to attach tubing to the inlet end 86 and the outlet end 88 of the indicator device 206, if desired, in order to direct a flow liquid disinfectant (not shown in FIG. 13 or FIG. 14) through the device. The data indicate that all of the (approximately 10⁶) spores in the indicator device were killed under the process conditions used in this Example.

### Example 2

### Use of Flow-Through Indicator Device to Monitor the Efficacy of a Liquid Disinfection Process

An indicator prepared according to Example 1 was placed into a mesh bag which was subsequently placed in the interior portion of a Medivators DSD 201 Automatic Endoscope Reprocessor (AER; Medivators, Inc., Minneapolis, Minn.) basin adjacent to an endoscope connected to the AER. The endoscope and the mesh bag containing the microbial indicator were contacted with a minimum effective concentration (0.35%) ortho-phthalaldehyde (OPA, RAPICIDE® OPA/28 High-Level Disinfectant; Medivators, Inc.) at 25 C in a standard disinfection process (5 minute OPA contact time at 25° C., followed by two rinses with purified water). In this Example, the indicator device was not connected (directly or indirectly) to the AER circulating pump system. At the end of the disinfection process, the spore carrier material was aseptically removed from the microbial indicator using sterile forceps and was transferred to a tube containing sterile malt extract broth, pH 7.5, containing glycine (0.025 mg/mL) and bromocresol purple (0.03 g/L). The broth was then incubated at 30° C. No growth in the broth after 7 days of incubation. Positive controls (i.e., spore-coated carriers that were not exposed to OPA contact) were also placed in tubes of malt extract broth and growth was evident (pH indicator changed from purple to yellow) within 7 day of incubation at 30° C.

### Example 3

### Use of Flow-Through Indicator Device Attached to a Flow System to Monitor the Efficacy of a Liquid Disinfection Process

Indicator devices were prepared according to Example 1. The Medivators DSD 201 Automatic Endoscope Reprocessor of Example 2 was used for all process tests. The malt extract broth of Example 2 was used to test the viability of spores after contact with the liquid disinfectant. Individual microbial indicators were connected to the flow pump system of the AER using tubing that was secured to the inlet and outlet of the indicator devices. Medivators Rapicide OPA (minimum effective concentration (0.35%)) maintained at 25 C was pumped through each indicator device at 100 rpm (approximately 25 ml/min). The indicator devices were contacted with the OPA for a total of 30 secs or 5 minutes, respectively. Duplicate indicator devices were tested for each contact time. Immediately after the contact period, each indicator device was disassembled and the spore carrier was aseptically transferred to malt extract broth and incubated up to 7 days as described in Example 3. A change from purple to yellow broth indicated positive growth of spores that survived contact with the liquid disinfectant. No growth was observed in tubes of malt extract containing spore carriers with spores that were contact with the OPA for either 30 seconds or 5 minutes at 25° C. Positive controls were tested as described for Example 2. The results indicated that all of the spores were killed on the spore carriers subjected to 30 second and 5 minute OPA contact times in the flow-through process. The positive controls showed positive growth within 7 days of incubation.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-47. (canceled)

48. An indicator device for verifying the efficacy of a liquid disinfection process, comprising:
a hollow body having at least one wall that forms a chamber, an inlet, an outlet, and a liquid flow path that extends from the inlet to the outlet;
a microorganism compartment disposed in the chamber; and
a plurality of indicator microorganisms enclosed in the microorganism compartment;
wherein the microorganism compartment has an interior volume that is defined in part by a first component that prevents unintentional passage of the indicator microorganisms out of the microorganism compartment via the inlet and a second component that prevents unintentional passage of the indicator microorganisms out of the microorganism compartment via the outlet.

49. The indicator device of claim 48, wherein the first component comprises a first microporous barrier.

50. The indicator device of claim 49, wherein the first microporous barrier comprises a membrane filter.

51. The indicator device of claim 48, wherein the second component comprises a second microporous barrier.

52. The indicator device of claim 50, wherein the first microporous barrier comprises a membrane filter.

53. The indicator device of claim 48, wherein the first component comprises a normally-closed one-way valve.

54. The indicator device of claim 48, further comprising at least one closure attached thereto, wherein the at least one closure is configured to prevent fluid flow through the inlet or the outlet.

55. The indicator device of claim 48, further comprising a detection medium contained in a frangible closed vessel disposed in the hollow body.

56. The indicator device of claim 48, wherein the plurality of indicator microorganisms comprises a plurality of vegetative cells.

57. The indicator device of claim 48, wherein the plurality of indicator microorganisms comprises a plurality of spores.

58. The indicator device of claim 57, wherein the indicator microorganism is of a genus selected from the group consisting of Aspergillus, Fusarium, Paecilomyces, Scedosporium, Trichophyton, and Penicillium.

59. The indicator device of claim 58, wherein the indicator microorganism is of a species selected from the group consisting of Aspergillus niger, Aspergillus brasiliensis, Aspergillus nidulans, Aspergillus oryzae, Fusarium solani, Fusarium verticillioides, Paecilomyces variotii, Scedosporium apiospermum, Trichophyton rubrum, and Penicillium camemberti.

60. The indicator device of claim 48, wherein the detection medium comprises an effective amount of at least one nutrient that facilitates germination or growth of the indicator microorganisms and/or, when the indicator microorganism is a spore, the effective amount of the at least one nutrient facilitates growth of a vegetative cell derived from the spore.

61. The indicator device of claim 61, wherein the detection medium comprises a detection reagent.

62. The indicator device of claim 61, wherein the detection reagent comprises an enzyme substrate for detecting an enzyme activity selected from a group consisting of xylanase, cellulase, α-galactosidase, β-galactosidase, β glucuronidase, phosphatase, α-glucosidase, β-glucosidase, amylase, glucose oxidase, laccase, and a combination of any two or more of the foregoing enzyme activities.

63. The indicator device of claim 48, wherein the plurality of indicator microorganisms is disposed on or in a microorganism carrier that is disposed in the microorganism compartment.

64. The indicator device of claim 48, wherein the hollow body consists essentially of a laminate of at least two films.

65. The indicator device of claim 64, wherein one film of the at least two films comprises a thermoplastic polymer.

66. The indicator device of claim 64, wherein one film of the at least two films consists essentially of a thermoplastic polymer.
